(19) United States
(12) Patent Application Publication
(10) Pub. No.: US 2007/0025874 A1
(43) Pub. Date: Feb. 1, 2007

(54) Water jet guided laser disinfection

(76) Inventor: Heiner Ophardt, Vineland (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 11/188,895

(22) Filed: Jul. 26, 2005

(51) Int. Cl. A61L 2/08; A61L 2/10; A61L 2/18; A61L 2/00

(52) U.S. Cl. 422/22; 422/28; 422/24; 422/292

(57) ABSTRACT

A method of disinfecting comprising directing a laser fluid jet through atmospheric air onto a surface, moving the jet relative the surface so that the jet directly impacts on substantially the entirety of a portion of the surface to be disinfected, the laser fluid jet comprising a stream of fluid and laser energy directed via the fluid stream.

### SCOPE OF THE INVENTION

This invention relates to disinfecting and, more particularly, to disinfecting surfaces by irradiation with laser energy directed to a surface via a stream of fluid.

### BACKGROUND OF THE INVENTION

Laser irradiation is used in a variety of ways for material processing, such as, cutting, drilling, welding and marking various materials including steel, steel alloys, non-ferrous metals, plastics and ceramics.

Waterjet guided lasers are known in which a laser beam is guided inside a low-pressure water jet onto a surface to be processed. For example, U.S. Pat. No. 5,902,499 to Richerzhagen teaches using a thin low-pressure water jet as a guide for a laser beam. The water jet passes through air such that an air-water interface is formed whereby water jet acts as a wave-guide to maintain the laser irradiation substantially within the laser beam. Reference is also made to U.S. Pat. No. 5,773,791 to Kuykendal which also teaches the laser embedded within a highly laminar high pressure water stream.

The present inventor has appreciated that conventional methods of hand washing and disinfecting hands and other items or surfaces thereof suffer the disadvantage that it is difficult to ensure adequate disinfecting. Many practical methods of disinfecting involve the use of chemicals and chemical solutions which after use requires disposal and therefore presents disposal and environmental problems. As well, insofar as human hands are to be washed, there is a significant problem in hands which are successively washed becoming sensitive to the chemicals and the skin of the hands becoming dry presenting discomfort as well as possible cracking and increased risk of infection.

The present inventor has appreciated that conventional disinfecting techniques involving the use of radiation are typically unduly severe as regard to the nature of the radiation involved or temperatures develop to be useful in irradiating a person's hands or other body surfaces.

### SUMMARY OF THE INVENTION

To at least partially overcome these disadvantages of previously known devices, the present invention provides a method and apparatus for disinfecting surfaces by directing a laser fluid jet onto a surface to be disinfected and moving the jet relative the surface so that the jet directly impacts substantially the entirely of a portion of the surface to be disinfected. The water jet is configured to act as a wave-guide for laser radiation directed substantially coaxially to the water jet. The water jet is directed at a pressure which does not due to its velocity injure the surface to be disinfected. The laser radiation to pass through the water jet is sufficient, on contact with the surface to be disinfected, such as the skin on a human hand, such as to provide a disinfecting function. The laser fluid jet is moved relative to the surfaces of a hand to be disinfected so as to not present at any location radiation beyond a maximum which might cause undue damage to the surface, such as a skin surface. Preferably, however, the radiation received at any portion of the surface is sufficient to heat and kill at least a portion of and, preferably, all microorganisms on the surface. Such absorption of energy by microorganisms is relatively prompt and immediate and heat generated thereby or by absorption of the laser energy by the surface such as skin is dissipated and reduced relatively quickly by the flow of fluid, preferably water of the fluid jet.

A preferred apparatus and method involves holding a person's hand above one or more nozzles directing a laser water jet on to the hand and moving the nozzle or nozzles in a manner such that the water jet will come to engage substantially the entirety of the surface of the hand to disinfect the same. The water from the water jet may be collected as in a catch basin and then disposed to a drain together with any removed materials such as dirt and microorganisms from the hand.

The method of disinfecting of the present invention is not limited for use on human hands but may be used on other live surfaces of a human body including wounds and internal surfaces to which access can be obtained for the laser fluid jet. The invention is not limited however to disinfecting by irradiating merely the surface of humans but can be used for disinfecting any other surfaces, such as, surfaces of surgical tools, kitchen surfaces, cleaning utensils or any other surfaces which may be desired to be disinfected. It will be appreciated that the intensity of the laser radiation and the velocity and/or size of the fluid jet may be modified having regard to the nature of the surface or surface to be disinfected. An advantage of the present invention is that the laser energy is directed via the fluid jet directly on to a surface to be disinfected. The fluid jet directs the laser energy onto the surface as a relatively constant flux effectively as a constantly focussed beam, wherever the fluid jet impacts. This is to be contrasted with requiring changing of the focus of a laser passed through air and onto a surface to provide a focussed air transmitted laser. Surfaces such as a hand have uneven contours and adjustment of an air transmitted laser would require constant, prompt refocusing to be effective. As well, as contrasted to energy losses in passing a laser through air, the relative energy loss of the laser radiation is low since the energy is effectively transferred via the fluid jet to the surface to be disinfected. When radiation in which radiation must pass through air in order to reach a surface to be disinfected, generally substantially larger radiation is required to be emitted such that adequate radiation might reach the surface to be disinfected.

Advances in computer technology such as, for example, in laser scanning techniques provide arrangements, which permit fast movement and re-direction of beams, jets and nozzles. As in a manner not dissimilar to that of an ink jet printer or a laser bar code scanner, various mechanical mechanisms may be provided so as to move the laser fluid jet relative the surface to be disinfected so that the jet is moved over paths so as to cumulatively directly impinge on substantially the entirely of any portion of the surface desired to be disinfected.

The cross-sectional area or size of the fluid jet is not limited other than to be selected so that the fluid jet provides a useful wave-guide for transference of the laser radiation. The laser jet may have a very small diameter, such as, 5 to 100 um, however, this is not necessary and provided the laser radiation is maintained within the jet and is distributed substantially uniformly over the cross-section of the jet, then the jet may be much larger sizes, for example, in the range of 1 mm to 500 mm or larger, such as 500 mm to 1 cm.

The movement of the laser jet relative the surface to be disinfected so as to impact substantially the entirety of a portion of the surface to be disinfected can adequately provide for disinfecting. The speed of movement of the laser jet across the surface to be disinfected can be of assistance in providing for transferral of relatively strong intensity laser irradiation without unduly harming any portion of the surface, such as, human skin or other body surface to be disinfected since the speed of movement of the jet across the surface will reduce the laser radiation irradiated on any area.

There is no particular limit as to the nature of the laser to be used. Many different conventional and commercially available laser emitting generators can be used. The laser may comprise laser energy emitted from a gas, diode or solid state laser. The radiation should be of any wavelength of radiation which provides the suitable disinfecting and which can be guided within the fluid jet. For example, for many forms of microorganisms, ultraviolet wavelength radiation is a preferred. It would be apparent that a person skilled in the art that in order to provide for disinfection of microorganisms on a surface to be disinfected then preferably over the area to be disinfected, there will be irradiation at acceptable radiation energy per unit area to kill the microorganisms. The desired levels of radiation and their effectiveness can be determined by simple experimentation. For example, with an argon fluoride (ArF) excimer laser, total laser beam fluence of between 8 J/cm² and 16 J/cm² are to be expected to kill many gram-negative bacteria.

The particular nature of the mechanism and/or nozzle for generating a suitable laser fluid jet is not limited.

Laserjet disinfection in accordance with the present invention has many uses, including not only the washing or disinfecting human hands but also washing and disinfecting various surfaces including surfaces which human hands come to contact such as, for example, surfaces to activate a soap dispenser, fingerprint readers and the like.

In accordance with the present invention, the laser beam radiated may be directed into a fluid jet by forming the fluid jet by passing fluid out of a nozzle from a pressurized fluid chamber with the laser radiation focussed as a beam coaxially through the nozzle from within the fluid chamber. The fluid jet emitted from the nozzle guides the laser beam by means of substantially total internal reflection at the fluid-air interface in a manner similar to conventional glass fibres. The fluid jet can thus be referred to as a fluid optical wave-guide of variable length. The fluid jet of course is essentially transparent for the laser beam. When the laser beam encounters the body to be disinfected, which absorbs the laser beam, the surface of the material to be deflected is heated to an extent that microorganisms on the surface are destroyed. The fluid which also engages on the surface aids in physically removing debris on other material on the surface to be disinfected and, as well, to remove any microorganisms.

Preferably, a pulsed laser is used so that a continuous fluid jet is able to immediately cool the surfaces being heated and thus reduce the temperature being reached. The pressure at which the fluid of the fluid jet is applied may be relatively low. Preferably, the wavelength of the laser radiation to be used are wavelengths that are limited to the range in which the absorption by the fluid, preferably water or alcohol or mixtures thereof, is low and preferably the wavelength has an absorption coefficient below one centimetre. Preferred wavelengths for the laser radiation are in the range of about 0.2 to 1.1 um.

The laser emitter or laser source may conveniently be coupled to the fluid jet or the nozzle which produces the fluid jet via an optical fibre which may, for example, end at a focussing lens.

The water pressure of the fluid jet may be selected to not injure the surface being disinfected. Preferably, relatively low pressure may be used such as in the range of 5 psi to 60 psi, more preferably, 10 psi to 50 psi including pressures which can be available from conventional building water taps without the need for special pumps.

Preferred water jets of relative low pressure and diameters in the laser ranges of, say, 1 mm to 1 cm or larger, may provide sufficient wave guides to reasonably consistently provide relative constant flux radiation on surfaces within a desired range of distances from the nozzle preferably in the range of 5 cm to 20 cm, to adequately disinfect for many applications, particularly with an ample flow of water.

### BRIEF DESCRIPTION OF THE DRAWINGS

Further aspects and advantages of the present invention will appear from the following description taken together with the accompanying drawings in which:

FIG. 1 is a side view of a simplified hand disinfecting apparatus in accordance with one embodiment of the invention;

FIG. 2 is a schematic perspective view of one embodiment of a turret mounted nozzle for a laser fluid jet in accordance with the present invention;

FIG. 3 is a cross-sectional side view through the nozzle of the laser fluid jet of FIG. 2;

FIG. 4 is a plan view of a surface to be disinfected showing paths of relative movement of the laser fluid jet.

FIG. 5 is a perspective view of a hand disinfecting apparatus in accordance with the present invention; and

FIG. 6 is a schematic, partially cross-sectional side view of the disinfecting apparatus of FIG. 5 showing dispensing onto a person's hands.

### DETAILED DESCRIPTION OF THE DRAWINGS

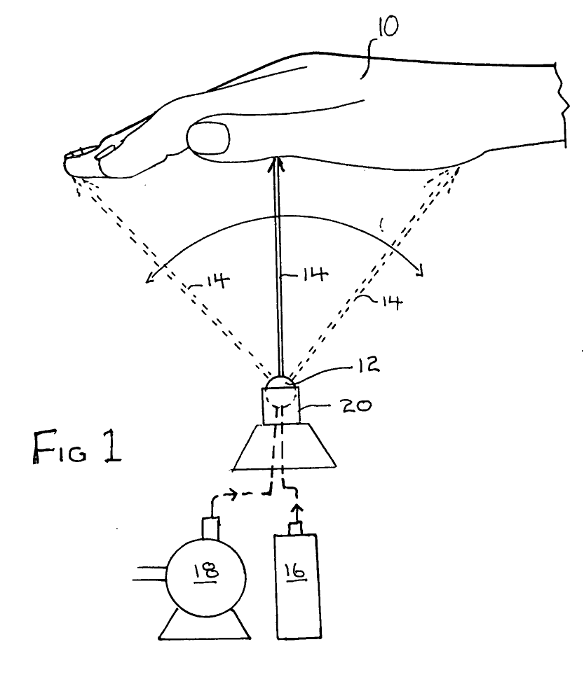

Reference is made to FIG. 1 which schematically illustrates a first embodiment of a disinfecting apparatus and method with a user's hand 10 disposed above a single nozzle 12 which directs a laser water jet 14 onto the under surface of the user's hand. Laser water jet 14 emanates from the nozzle 12 which is provided with laser radiation from a laser 16 and pressurized water from a water pump 18. The nozzle 12 is schematically illustrated as mounted within a turret support 20 as a ball-like member journalled in the turret support 20 for movement about two axes so as to direct the laser water jet 14 in different directions as, for example, indicated by the laser water jet 14 being both shown in solid and dotted lines in FIG. 1 in different positions. By relative movement of the turret nozzle 12, the laser water jet 14 is moved relative to the user's hand. The laser water jet can by such movement be caused to impact substantially the entirety of the under surface of the hand 10 which is desired to be disinfected.

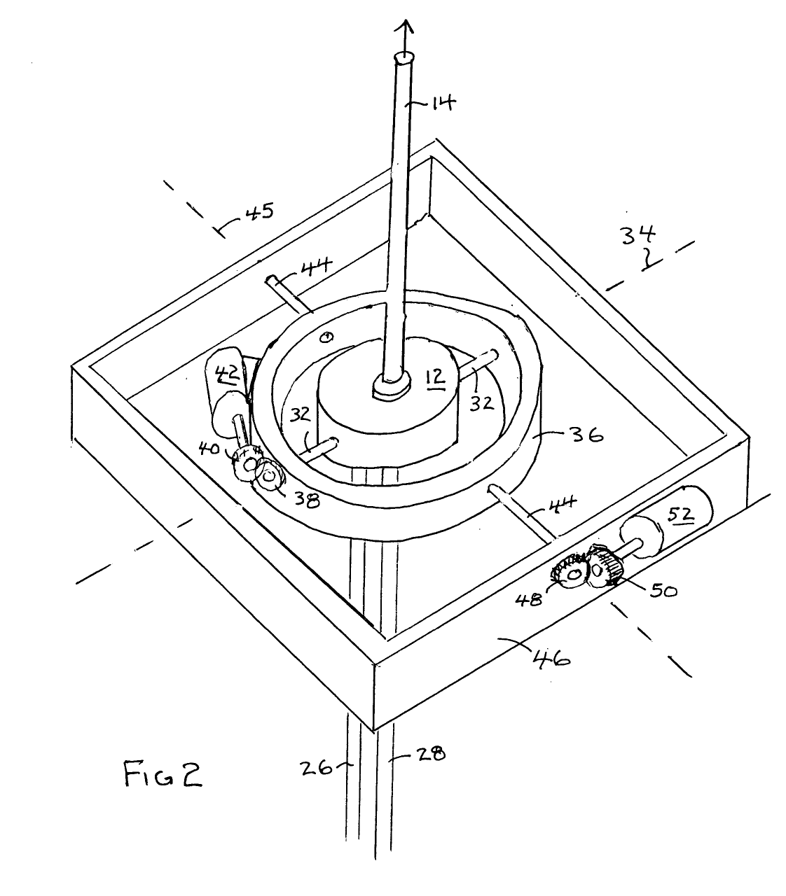

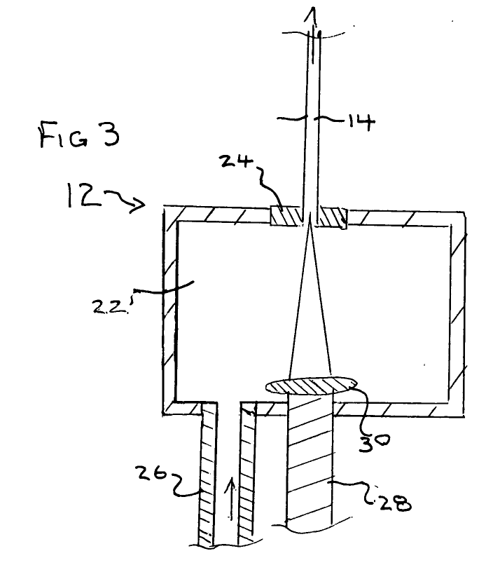

Reference is made to FIG. 2 which illustrates a schematic arrangement for providing a turret mechanisms by which the nozzle 12 can be moved to different orientations as pivoted about two different axes. FIG. 2 shows a generally cylindrical nozzle with a short length of a laser fluid jet 14 being discharged therefrom. The nozzle 12 is shown in cross-section in FIG. 3 as comprising an enclosed water pressure chamber 22 having a nozzle outlet 24 from which the laser water jet 14 is shown being dispensed. Water is provided into the chamber under pressure via a flexible water inlet tube 26 as from the water pump 18 of FIG. 1. A flexible optical fibre 28 enters the chamber 22 in a sealed relation. Laser radiation is carried via the optical fibre 28 from a laser such as laser 16 in FIG. 1. The radiation passes through a lens 30 which focuses the laser radiation into the centre of the outlet of the nozzle outlet 24 and hence into the water jet being discharged from the chamber 22. The laser water jet 14 provides a thin cylindrical column of water with a side wall having an interface with air so as to provide a wave-guide for the laser radiation and thus transmits the laser radiation through the water jet to, concurrently with the water of the jet, impact on the surface of the hand 10 to be disinfected.

Referring to FIG. 2, the nozzle 12 is shown with the nozzle water jet 14 disseminating upwardly therefrom. The nozzle 12 is fixed on first axles 32 disposed about a first axis 34. The first axles 32 pass at diametrically opposite locations through a circular inner support ring 36 and are journalled to the inner support 36 for relative rotation. The first axles 32 carry a first driven gear 38. The first driven gear 38 is coupled via a first drive gear 40 to a first motor 42 mounted to the inner support circular ring 36. The first motor 42 is preferably a step motor which can be electronically controlled to precisely rotate the first driven gear 38 and thereby the nozzle 12 about the first axis 34 relative the inner support ring 36.

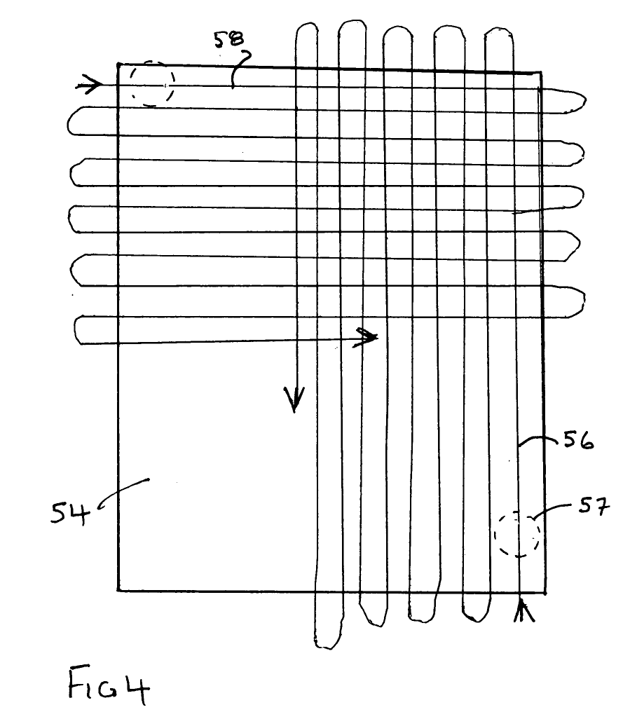

The inner support ring 36 is fixed on second axles 44 disposed about a second axis 45. The second axels 44 pass at diametrically opposite locations through a second outer support ring 46 and are journalled to the outer support ring 46 for relative rotation. The second axles carry a second driven gear 48. The second driven gear 48 is coupled via a second drive gear 50 to a second motor 52 mounted to the outer support ring 46. The second motor 52, also is preferably a step motor which can be electronically controlled to precisely rotate the second driven gear 48 and thereby the inner support ring 36 about the second axis 45 relative the outer support ring 46. Both the water inlet tube 26 and the optical fibre 28 are provided to be sufficiently resilient to permit the nozzle 12 to be moved to different relative varying angulations about the two axes 34 and 45 such that the laser water jet 14 can be directed onto the under surface of a hand to follow various patterns as illustrated, for example, in FIG. 4 to be moved and cumulatively impact on the entirety of the portion of the surfaces of the hand to be dispensed.

this regard, reference is made to FIG. 4 which illustrates a rectangular portion 54 of the under surface of the hand 10 which is desired to be disinfected. A first path indicated as 56 indicates a series of back and forth parallel lines representing a serpentine manner in which the water laser jet 14 is moved. The laser water jet 14 is to have a diameter at impact on the surface as shown, for example, as a circle 57 in dashed line. When moved along the serpentine path 56, the laser water jet provides radiation over the entire surface of the rectangle, preferably with the area upon which radiation is radiated in each straight run of the path over the lapping at least to some extent with the area upon which radiation is radiated in parallel adjacent run of the path. A second path 58 of parallel lines shows another path along which the laser jet may preferably also be moved relative to the surface after the first path to further ensure the jet directly impacts on the entirety of the surface to be disinfected. While FIG. 4 illustrates movement of laser jet in a path of parallel lines, any pattern for the path of movement of the laser jet may be adopted provided that at the end of the path, substantially the entire surface to be disinfected has been impacted by the laser jet. Other preferred patterns can include spirals from a centre point. Of course, a plurality of different patterns could be used in sequence.

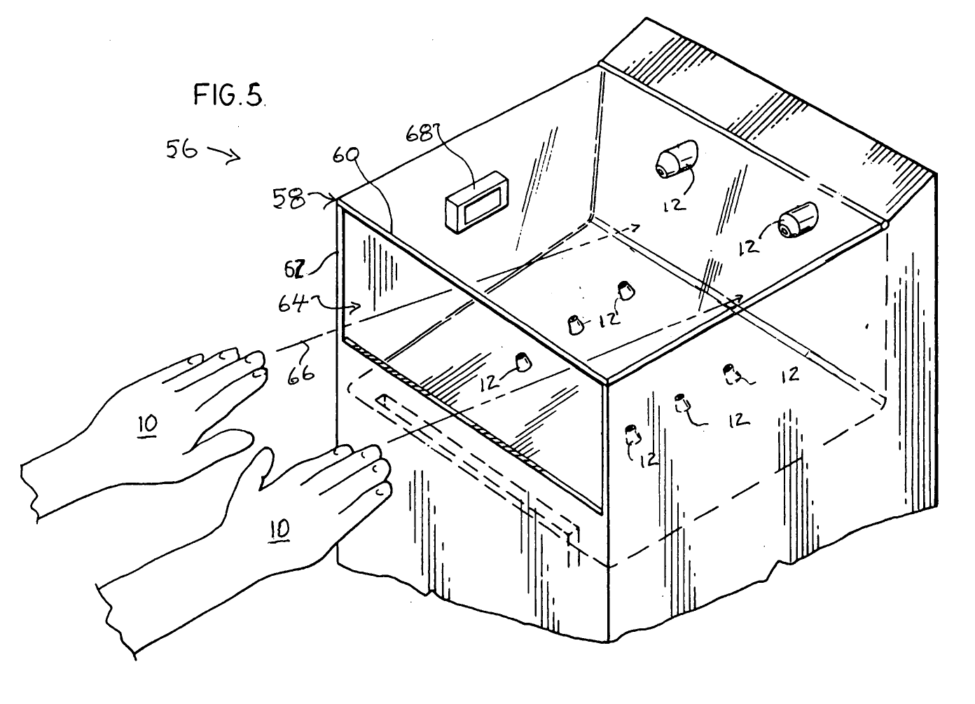

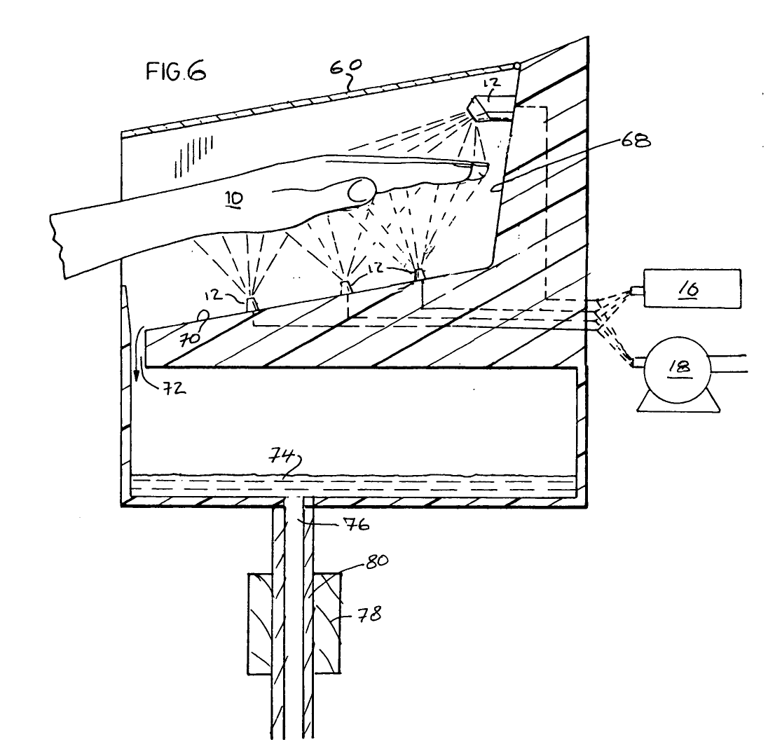

Reference is made first to FIGS. 5 and 6 showing a hand disinfecting apparatus 56 providing the substantially enclosed compartment 58 to which a user's hands 10 are to be placed for disinfecting. The enclosure 56 is formed by a shroud having a transparent top 60 and sides 62 to contain over sprays and assist a user in holding his arms in a desired orientation for disinfecting. The shroud has an open front opening 64 through a person's hands 10 are to be directed as along the arrows indicated 66. A sensor 68 is provided inside the enclosure to sense the presence of a user's hands inside the closure and activate the disinfecting apparatus. A plurality of nozzles 12 are provided inside the enclosure to direct laser water jets onto the surfaces of the hands to be disinfected. In the preferred embodiment, the enclosure has a back support wall 68 and a lower support surface 70. For each hand, one nozzle 12 is mounted on the back wall 68 and three nozzles 12 are shown mounted on the lower support wall 70. A control mechanism may be provided to control the timing and movement of the jets so as to reasonably ensure that the jets would spray onto the entirety of a user's hand. The control mechanism can give signals and directions to a user regarding placement of their hands, and for start up and finishing of a washing cycle. The lower support wall 70 is angled forwardly and carries a drain opening 72 at its forward end so as to permit water from the laser jet nozzles after engaging the hands to flow downwardly under gravity as to an optional catch basin 74 and then out a drain 76. The catch basin can be used in the event the fluid is desired to be reused or recycled as may be advantages, as for example, when the fluid being directed through the nozzles may be, for example, alcohol rather than water. A separate commercially available UV radiation disinfector 78 may be provided to farther disinfect fluid exiting the drain as by passing UV radiation through a transparent portion of a drain pipe 80.

FIG. 6 schematically illustrates a laser 16 which is a source of laser radiation and a pump 18 which provides pressurized fluid. FIG. 6 schematically illustrates the radiation from the laser 16 and the fluid from the pump 18 being coupled together by various lines shown in dotted lines such that laser radiation and pressurized fluid is provided to each of the nozzles 10.

The preferred embodiments illustrate the direction of a laser fluid jet onto a person's hand for disinfecting and cleaning the same. It is to be appreciated that other disinfecting apparatuses can be provided with one or more laser fluid jets providing a jet to impact on surfaces in any manner which are desired to be disinfected. Many versions of the apparatus will occur to a person skilled in the art. As one example, the laser jet could dispense from a hand held wand which could be used as in medical applications, for example, to disinfect around sounds, cuts, abrasions and the like.

Many modifications and variations will now occur to persons skilled in the art. For a definition of the invention, reference may be made to the appended claims.

1. A method of disinfecting comprising:
directing a laser fluid jet through atmospheric air onto a surface, moving the jet relative the surface so that the jet directly impacts on substantially the entirety of a portion of the surface to be disinfected,
the laser fluid jet comprising:
a stream of fluid and laser energy directed via the fluid stream.

2. A method as claimed in claim 1 wherein the fluid is selected from the group comprising water and alcohol.

3. A method as claimed in claim 1 wherein the fluid stream acts as a wave-guide to transmit the laser energy internally therethrough.

4. A method as claimed in claim 1 wherein said laser energy is selected from light energy emitted from a gas, diode or solid-state laser.

5. A method as claimed in claim 4 wherein said laser energy is pulsed.

6. A method as claimed in claim 3 wherein said fluid stream is generated by passing the fluid through a nozzle to provide a continuous column of fluid of substantially uniform cross-section, and
the laser energy comprises a laser beam extending substantially parallel to the column of fluid through the column of fluid.

7. A method as claimed in claim 4 wherein said laser energy includes ultraviolet wavelength radiation.

8. A method as claimed in claim 1 wherein said jet provides laser energy onto the surface with sufficient total fluence to substantially kill any microorganisms thereon.

9. A method as claimed in claim 8 wherein the jet is moved relative the surface so that the portion of the surface to be disinfected receives ultraviolet radiation in the range of about 8 to 16 J/cm².

10. A method as claimed in claim 8 wherein the surface to be disinfected is a part of a living human or animal.

11. A method as claimed in claim 1 wherein the surface to be disinfected is a surface of a hand of a human.

12. A method as claimed in claim 11 including the first step of holding a person's hand at desired location relative to a nozzle from which the jet emanates and maintaining the hand at a relatively constant position relative to the nozzle for a desired period of time, while moving the nozzle to direct the jet to impinge substantially the entirety of the surface of the hand directed toward the nozzle.

13. A method as claimed in claim 12 including a first nozzle to direct a first laser fluid jet onto an upper surface of the hand and a second nozzle to direct a second laser fluid jet onto a lower surface of the hand.

14. A hand washing apparatus comprising a nozzle directing a laser fluid jet through atmospheric air into contact with a hand, wherein the laser fluid jet comprises a stream of fluid and laser energy directed via the fluid stream acting as a waveguide.

15. An apparatus as claimed in claim 14 wherein the jet is moved relative the hand to contact substantially the entirety of the surfaces of the hand directed toward the nozzle.
